(19) United States
(12) Patent Application Publication
(10) Pub. No.: US 2024/0091036 A1
(43) Pub. Date: Mar. 21, 2024

(54) A STENT FOR IMPLANT WITHIN A VESSEL

(71) Applicant: Venous Stent B.V., Maastricht (NL)

(72) Inventors: Cornelis Hendrikus Anna WITTENS, Maastricht (NL); Bettina Dimmler, Maastricht (NL)

(21) Appl. No.: 18/039,652

(22) PCT Filed: Dec. 8, 2021
(86) PCT No.: PCT/NL2021/050745

§ 371 Date: May 31, 2023

(30) Foreign Application Priority Data
Dec. 8, 2020 (WO) PCT/NL2020/050765

(51) Int. Cl. A61F 2/966

(52) U.S. Cl. CPC A61F 2/966; A61F 2002/828

(57) ABSTRACT

The invention relates to stents, in particular to a stent assembly for insertion in a vessel of a human or animal body.

The invention also relates to a catheter stent insertion device for inserting a stent assembly according to the invention in a vessel of a human or animal body.

The invention also relates to a method for inserting a stent assembly according to the invention in a vessel of a human or animal body using a catheter stent insertion device according to the invention.

### FIELD OF THE INVENTION

The invention relates to stents, in particular to a stent for insertion in a vessel of a human or animal body.

The invention also relates to a catheter stent insertion device for inserting a stent according to the invention in a vessel of a human or animal body.

The invention also relates to a method for inserting a stent according to the invention in a vessel of a human or animal body using a catheter stent insertion device according to the invention.

### BACKGROUND OF THE INVENTION

Stents are widely used in medicine to keep the passageway open of the lumen of an anatomic vessel or duct. There is a wide variety of stents used for different purposes, from expandable coronary, vascular and biliary stents, to simple plastic stents used to allow the flow of urine between kidney and bladder.

In particular, vascular stents are commonly placed in arteries and veins in order to support the affected, weakened vessel wall of the artery or vein, as part of angioplasty. The commonly used procedure of treatment implements a catheter to insert and guide an expandable stent towards the site of the weakened vessel wall. The catheter containing the compressed peripheral stent is hereto inserted into an artery or vein and guided using suitable imaging techniques, such as fluoroscopy, towards the site of deployment. Once the catheter is properly positioned the compressed stent is deployed and expanded against the inner vessel wall of the artery or vessel.

Because of the external compression and mechanical forces subjected to these locations, flexible stent materials such as nitinol are used in a majority of peripheral stent placements.

Segmental stents being composed of at least two individual interconnected stent segments with a high radial force and high flexibility can be implemented within vessels. A problem associated with such stents being assembled of multiple stent segments is that once the most proximal stent segment is being deployed from the catheter stent insertion device at the desired or intended deployment location within the vessel, the deployed stent segment immediately will expand into its expanded configuration and come in abutment with the inner vessel wall of the vessel.

In general, when considering stenting a diseased vein, it is not possible to measure the exact length needed for a venous stent with pre-intervention ultrasound, CT of MR imaging techniques. It is only during the stent procedure that with phlebography and intravascular ultrasound (IVUS) techniques, the exact length of the stent can be identified. The patency of a stent is mainly depending on the exact positioning of the stent to guarantee good in and outflow, improving the symptoms of patients and the patency of the stent.

Any retraction of the deployed and expanded stent segment back into the catheter stent insertion device for repositioning purposes within the vessel, for example due to a deployment (and expansion) on an incorrect or non-preferred position within the vessel, is no longer possible. The position of an already deployed and expanded first stent segment within the vessel cannot be corrected and the remainder of the stent assembly yet accommodated in the catheter stent insertion device has to be deployed entirely within the vessel. This may lead to a stent being deployed and expanded, which does not fully support the affected vessel wall of the vessel over its intended length and as such the stenting procedure may not be considered successful.

### DESCRIPTION OF THE INVENTION

The invention aims to provide a solution for the above identified problem, allowing the easier correction of the ultimate longitudinal deployment position of the stent assembly within the vessel, whilst guaranteeing a proper support of the affected vessel wall over its full length by the deployed and expanded stent.

According to the invention a stent assembly for insertion in a vessel of a human or animal body is proposed, said stent assembly having a proximal end, a distal end and a longitudinal stent axis, and comprising at least two stent segments, as well as segment interconnecting means interconnecting two stent segments, wherein said segment interconnecting means are structured in adjusting a distance between said two stent segments within the vessel between a first configuration, wherein said distance within the vessel is minimal and a second configuration, wherein said distance within the vessel is maximal, due to a translation displacement along said longitudinal stent axis of one of said two stent segments relative to the other of the two stent segments within the vessel.

Adjusting the length of the stent assembly with respect of the intended location of deployment within the vessel through translation displacement along the longitudinal stent axis of the stent segments relative to each other can be achieved in a more direct yet versatile manner and with an improved accuracy compared to existing stent designs. With such stent assembly design which can be deployed and extended through translation displacement of the individual stent segments, a proper support of the affected vessel wall over its full stent length is achieved.

Accordingly, the in length adjustable stent assembly according to the invention is capable of covering the complete ilio-femoral tract in a human or animal being, with the application/insertion of only one stent assembly. This has a significant benefit compared to already known stent placement procedures, wherein two separate stents are needed resulting in an unfavorable overlap. Accordingly, as the length of the stent assembly according to the invention can be adjusted with respect of the intended location of deployment within the vessel, the adjustment and positioning of this in-situ in length adjustable stent assembly can be performed more precise using the phlebography and IVUS information collected during the procedure. In an example of the stent assembly according to the invention, for interconnecting said at least two stent segments, said segment interconnecting means comprise at least one elongated filament rod having two rod ends, a first rod end being connected to the first stent segment and the second rod end being connected to the second stent segment, wherein a distance between both first and second rod ends is smaller in said first configuration than in said second configuration.

Thus, the stent segments are always properly interconnected, and the coherence or unity of the overall stent assembly is maintained. As the distance within the vessel between both first and second rod ends is smaller in said first configuration than in said second configuration the filament rods allow for the length adjustment of the stent within the vessel due to the translation displacement of the stent segments relative to each other and being interconnected with said filament rods.

In a first example, in said first configuration and said second configuration said first rod end and said second rod end are radially offset with respect to each other, whereas in another example of a stent assembly according to the invention, in said first configuration and said second configuration said first rod end and said second rod end are longitudinally aligned with respect to each other, seen in the direction of said longitudinal stent axis. Both examples guarantee a secure interconnection between adjacent stent segments, thus maintaining the overall coherence of the segmental stent, and allow for a versatile translation elongation of the stent.

Preferably, said at least one elongated filament rod is structured to extend in length, thus ascertaining the translational displacement of the stent segments relative to each other and as a result the overall translation elongation of the stent assembly upon deployment in a vessel.

In particular, said at least one elongated filament rod is structured to irreversible extend in length. Herewith it is ascertained that the translation elongation of the stent assembly after deployment in the vessel is permanent and that an undesired returning of the elongated stent inside the vessel towards a stent configuration with a shrunk or diminished length is prevented. As the segmental stent contacts the vessel wall after deployment and expansion of the several stent segments, with the irreversible length extension aspect of the elongated filament rod it is avoided that damage to the inner vessel wall is induced, which would be the case in the event that the deployed and expanded stent assembly shrinks seen in its longitudinal stent axis within the vessel.

In an advantageous example, said at least one elongated filament rod is manufactured from an extendable material, for example from a flexible material. In yet another examples, which also allows for a versatile translational displacement of the adjacent stent segments, said at least one elongated filament rod has a telescopic structure or a zigzag structure or a coil structure.

Furthermore, in yet another example of a stent assembly according to the invention, which is capable of supporting or stenting larger lengths of affected vessel walls, or even may be implanted in meandering vessel parts, the stent comprises a proximal stent segment, a distal stent segment and one or more intermediate stent segments disposed between the proximal and distal stent segments, and wherein said segment interconnecting means interconnect each of said stent segments.

In this example furthermore said proximal stent segment has a first length, said distal stent segment has a second length, and said intermediate stent segments have a third length, all seen along said longitudinal axis of the stent, wherein said third length is smaller than said first and second length, and in particular said third length is 5-15 mm. As such the stent assembly has a proximal stent part as well as a distal stent part of a longer length that the individual intermediate stent segment. The longer stent length at its proximal and distal end part serves as a proper support for the affected vessel wall at the beginning and the end of the expanded stent within the vessel. This guarantees a proper and stable anchoring of the stent within the vessel.

In this simplified example said first length and said second length are the same, whereas in another example said first length is longer than said second length, in particular said first length is 30-50 mm and said second length is 10-30 mm.

In yet another example said maximal distance between said stent segments is 1-20 mm.

Depending on the stenting procedure to be performed on the affected vessel of the patient, the number of said intermediate stent segments is between 1-30.

An example of a catheter stent insertion device for inserting a stent assembly composed of at least two stent segments according to the invention in a vessel of a human or animal body is proposed, which catheter stent insertion device allows for adjusting the length of the stent assembly with respect of the intended location of deployment within the vessel, such that the affected vessel wall always is properly supported over its full length by the adjusted stent after deployment and expansion in the vessel.

Hereto the catheter stent insertion device according to the invention at least comprises a hollow stent accommodating tube having an open proximal tube end and a distal tube end, said hollow stent accommodating tube being arranged for accommodating said stent assembly in a compressed configuration, as well as lengthening means arranged in adjusting within the hollow stent accommodating tube a distance between an already deployed stent segment and a next interconnected stent segment between a first configuration, wherein said distance is minimal and a second configuration, wherein said distance is maximal, by translating the next interconnected stent segment along the longitudinal stent axis relative to the already deployed stent segment.

This allows for the translational adjustment the length of the stent with respect of the intended location of deployment within the vessel, such that the affected vessel wall always is properly supported over its full length by the adjusted stent after deployment and expansion in the vessel.

In an example of the catheter stent insertion device, said lengthening means comprises a translation spindle extending through the stent assembly in its compressed configuration, said translation spindle comprising an enlarged distal stent engagement end having an outer dimension larger than an outer dimension of a stent segment in its compressed configuration.

Herewith the distance between still not-yet deployed and compressed stent segments can be set by pulling at the not-yet deployed stent segment in a distal translational direction. As the enlarged distal stent engagement end has an outer dimension smaller than an inner dimension of a stent segment in its expanded, deployed configuration, this allows a simple yet repetitive lengthening step with a next not-yet deployed and compressed stent segment.

Preferably guidance means for guiding said hollow stent accommodating tube with its proximal tube end towards a deployment location within said vessel.

The invention also relates to a method for inserting a stent according to the invention at a deployment location within a vessel of a human or animal body using a catheter stent insertion device according to the invention, the method comprising the steps of:
A inserting the catheter stent insertion device accommodating said stent composed of at least two stent segments in a compressed configuration in said hollow stent accommodating tube with its open proximal tube end in the vessel;
B guiding the catheter stent insertion device towards said deployment location within the vessel;
C retracting said hollow stent accommodating tube until the proximal one of the at least two stent segments is deployed via the open proximal tube end in the vessel;
D retracting one of said at least two stent segments along the longitudinal stent axis relative to the other already deployed one of the at least two stent segments, thereby adjusting a distance between said at least two stent segments between a first configuration, wherein said distance is minimal and a second configuration, wherein said distance is maximal;
E retracting said hollow stent accommodating tube and deploying the next one of the at least two stent segments via the open proximal tube end in the vessel.

In particular the method is further characterized by the step of:
D1 repeating step D for each next individual stent segment of the stent.

In yet another example, in the method steps D and D1 are performed for each of the individual stent segments within the hollow stent accommodating tube prior to step C.

### BRIEF DESCRIPTION OF THE DRAWINGS

The invention will now be described in more details in reference to the accompanying drawings, which drawings show in:

FIG. 1a-1d a schematic first example of a stent assembly according to the invention in several states of translational expansion;

FIG. 2a-2d a schematic second example of a stent assembly according to the invention in several states of translational expansion

FIG. 3a-3d other schematic examples of a stent assembly according to the invention;

FIGS. 4a-4b-4c detail view of the stent assembly of FIGS. 1-3 in several expansion states;

FIG. 5a-5e a schematic example of a catheter stent insertion device according to the invention.

### DETAILED DESCRIPTION OF THE INVENTION

For a better understanding of the invention like parts in the drawings are to be denoted with like reference numerals.

In the detailed description below as well as in the claims various parts are denoted with the classification “proximal” and “distal”. These classifications are to be considered in relation to the location of the heart of the human or animal subject in which the stent is to be implanted. Hence the classification “proximal” is to be understood as meaning “closest to the heart” or “in a direction towards the heart”. Similarly, “distal” is to be understood as meaning “farthest from the heart” or “in a direction away from the heart”.

In general, when considering stenting a diseased vein, it is not possible to measure the exact length needed for a venous stent with pre-intervention ultrasound, CT of MR imaging techniques. It is only during the stent procedure that with phlebography and intravascular ultrasound (IVUS) techniques, the exact length of the stent can be identified. The patency of a stent is mainly depending on the exact positioning of the stent to guarantee good in and outflow, improving the symptoms of patients and the patency of the stent.

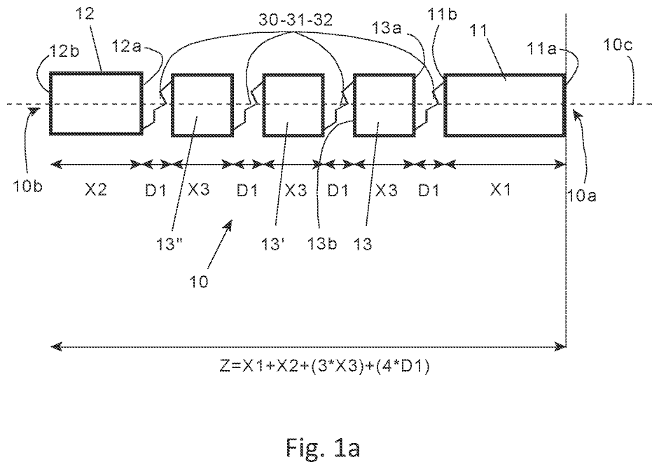

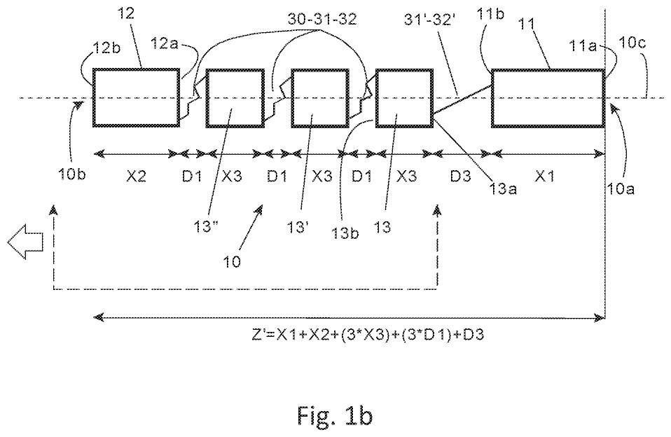

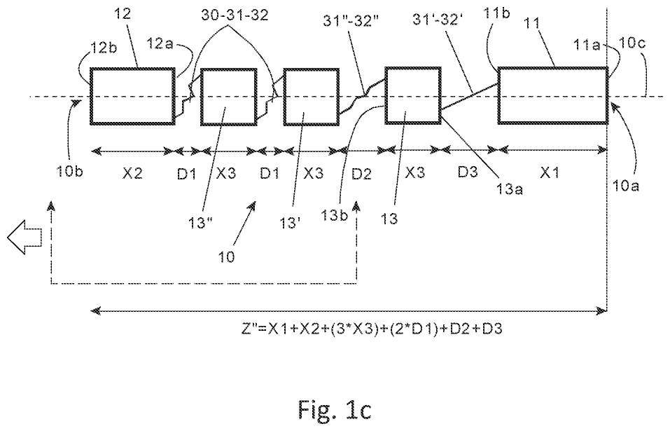

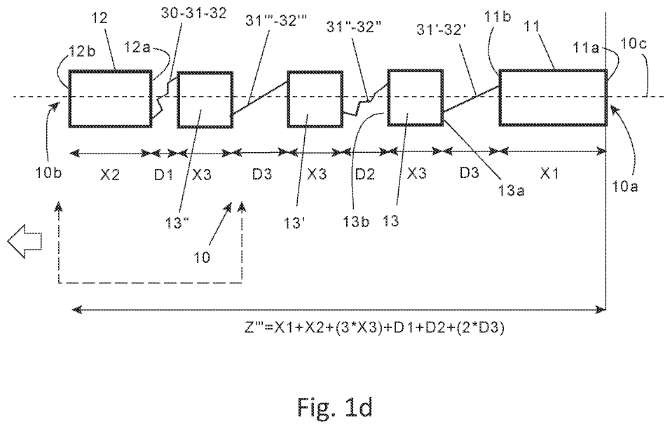

In order to address the above stenting length adjustment problem, in FIGS. 1(1a-1d), 2(2a-2d), 3(3a-3d) and 4, several different examples of a stent assembly according to the invention are shown, in several states of expansion in translation direction. In these Figures the stent assembly is denoted with reference numeral 10.

Stent assembly 10 comprises a proximal stent segment 11 with a proximal segment face 11a, which corresponds with the proximal stent end 10a of the complete stent assembly 10. The proximal stent segment 11 also has a distal segment face 11b. The stent assembly 10 also comprises a distal stent segment 12, which in a similar fashion is provided with a proximal segment face 12a and a distal segment face 12b, the latter also forming the distal stent end 10b of the stent assembly 10.

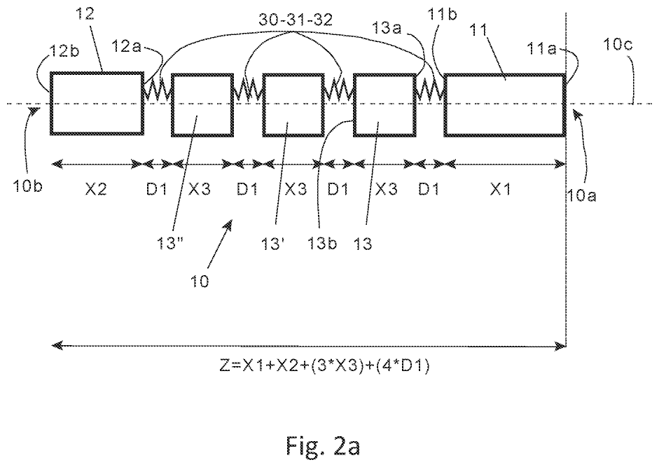

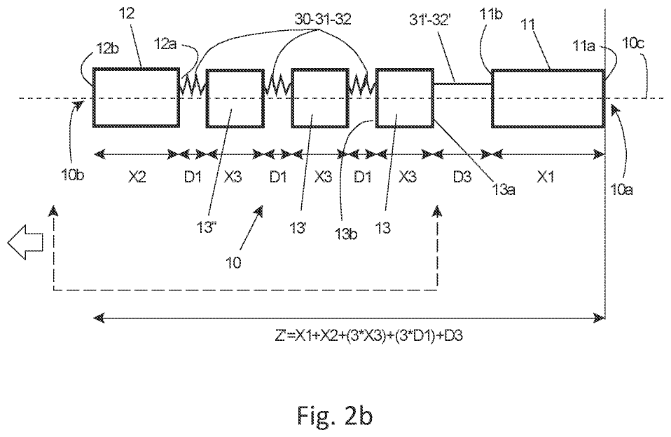

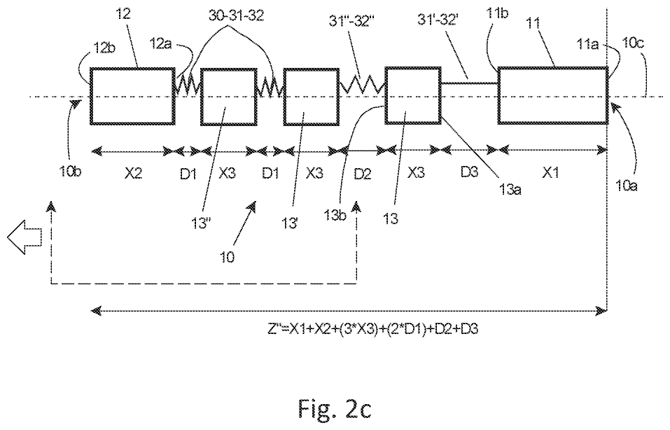

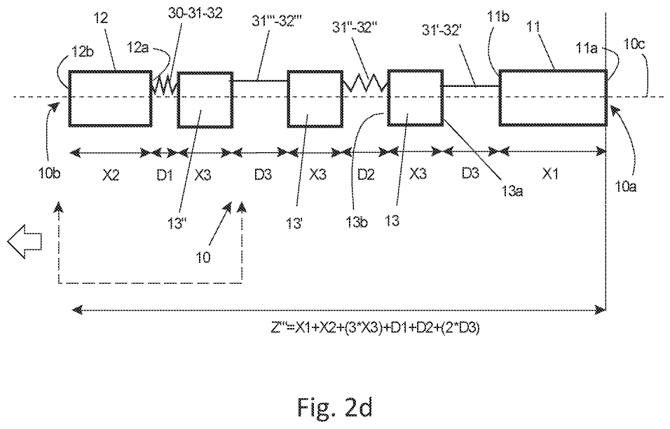

Between the proximal stent segment 11 and the distal stent segment 12, several intermediate stent segments 13, 13′ and 13″ are accommodated. It is to be noted that the number of intermediate stent segments can be arbitrarily chosen. Next to the embodiments as shown in FIGS. 1a-1d and 2a-2d, which depicts three intermediate stent segments 13-13′-13″, any arbitrary number of intermediate stent segments 13 (one, two, three, four, . . . till 30 or more) can be chosen, depending on the stent implant application to be performed.

In this example, the intermediate stent segments 13-13′-13″-etc. are identical in terms of shape and dimensions. However, this equal configuration is not required for the functionality of the stent 10 according to the invention.

The first embodiment of the stent assembly 10 depicted in the FIGS. 1a-1d as well as the second embodiment shown in FIGS. 2a-2d is accommodated in a compressed configuration in a catheter stent insertion device with the individual compressed stent segments 12-13″-13′-13-11 (seen from the distal stent end 10b towards the proximal stent end 10a of the stent 10) all being positioned at a minimal distance D1 (theoretically 0 (zero) mm, but in practice around 0.1-1.0 mm) from each other. The proximal stent end 10a forms the frontal part of the stent assembly 10, seen in relation to the orientation of the heart of the human or animal body.

When the catheter stent insertion device is inserted into the vessel with the stent assembly 10 accommodated in its compressed configuration, the proximal stent segment 11 is to be inserted and deployed as the first segment at the desired location within the vessel of a human or animal body, the initial length X1 of the proximal stent segment 11 needs to be sufficiently long, allowing for a partial, incomplete insertion and deployment of the proximal stent segment 11 into the vessel and checking of its correct position within the vessel using suitable known imaging techniques, such as fluoroscopy, and a subsequent retraction of said partially deployed proximal stent segment 11 back into the catheter stent insertion device in case of an incorrect position being observed.

Preferably the length X1 is such that a partial deployment of the first, proximal stent segment 11 within the vessel over approximately an insertion/deployment length corresponding with 50% of X1 still allows for a proper retraction of said partially deployed proximal stent segment 11 back into the catheter stent insertion device and a subsequent repositioning of the (proximal end of the) catheter stent insertion device within the vessel for a renewed, now correct deployment of the proximal stent segment 11.

In this example the individual stent lengths X1 of the proximal stent segment 11) and X2 (being the length of the distal stent segment 12) are both larger than the individual stent length X3 of the intermediate stent segment (either 13-13′-13″). For example, X1 and X2 are of an identical length, whereas in the FIGS. 1a-1d and 2a-2d it is shown that X1 is larger than X2. Typical dimensions for X1, X2 and X3 are: X1 between 30-50 mm, X2 between 10-30 mm and X3 between 5-15 mm. A typical diameter of all stent segments, hence the overall stent assembly 10 is between 8-35 mm.

The stent assembly 10, as depicted in FIG. 1a as well as in FIG. 2a, is shown in its initial, e.g. its shortest, configuration within the catheter stent insertion device, meaning that the stent assembly 10 has a minimal length, measured from its proximal stent end 10a until its distal stent end 10b. Said minimal length is denoted with the reference numeral Z. The initial overall length Z of the stent assembly 10 as denoted in FIGS. 1a and 2a is composed of (the summation of) the individual stent length X1 (of the proximal stent segment 11), X2 (the length of the distal stent segment 12), three times the individual stent length X3 of the three intermediate stent segments 13-13′-13″, as well as four times the minimal distance D1 present between each adjacent stent segment.

In FIGS. 1a-1d and 2a-2d interconnecting means 30 are present, which interconnect the proximal stent segment 11 with the first intermediate stent segment 13, interconnect the first intermediate stent segment 13 with the second intermediate stent segment 13″, interconnect the second intermediate stent segment 13″ with the third intermediate stent segment 13′″, etc. etc. and finalize interconnect the final, here the third intermediate stent segment 13—with the distal stent segment 11.

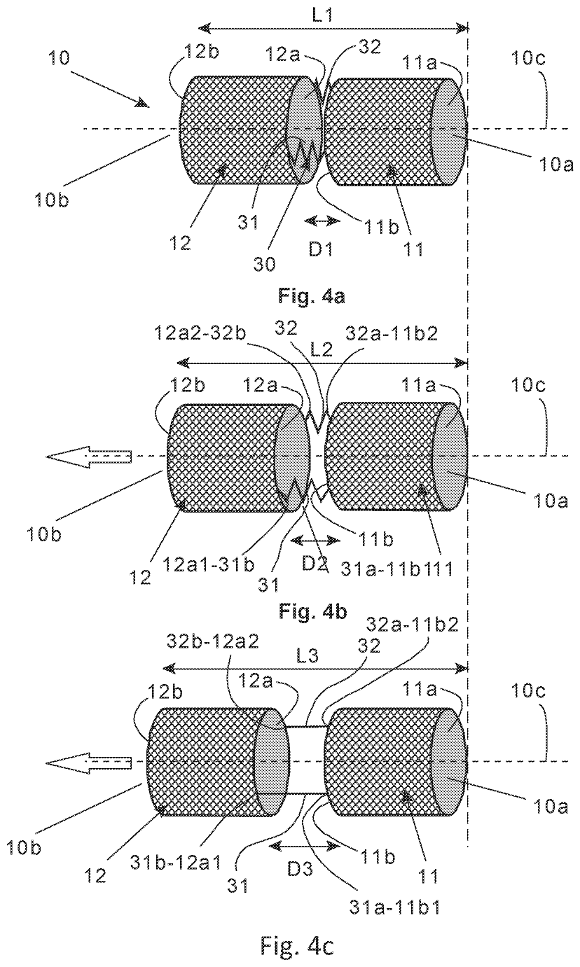

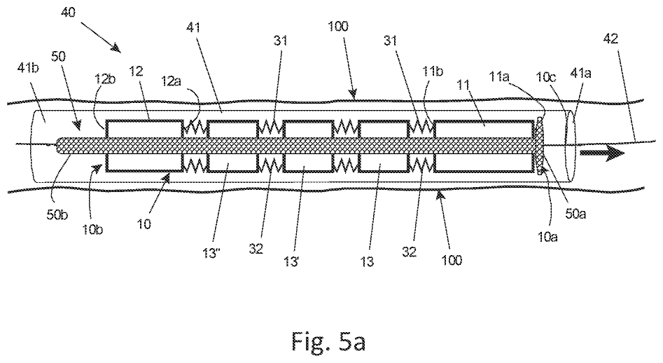

In both embodiments depicted in FIGS. 1a-1d and 2a-2d (and in FIGS. 4a-4c) the interconnecting means 30 are composed of the several elongated rod elements 31-32, each having a first, proximal rod end 31a (32a) and a second, distal rod 31b (32b) end interconnecting adjacent stent segments. The several elongated rod elements 31-32, (31′-32′), (31″-32″) (31′″-32′″) are structured to extend in length. Preferably those elongated rod elements 31-32, (31′-32′), (31″-32″) (31′″-32′″) are structured to irreversible extend in length, e.g. by means of plastic deformation. Herewith the translational distance between adjacent stent segments can be amended and set at a desired permanent intermediate distance X1, X2 or X3 depending on the desired overall length of the stent assembly 10 within the vessel.

Preferable, the elongated filament rod elements 31-32 (31′-32′) (31″-32″) (31′″-32′″) are manufactured from an extendable material, for example from a flexible material, or the elongated filament rod element have in another example a telescopic structure also allowing extension in its longitudinal direction.

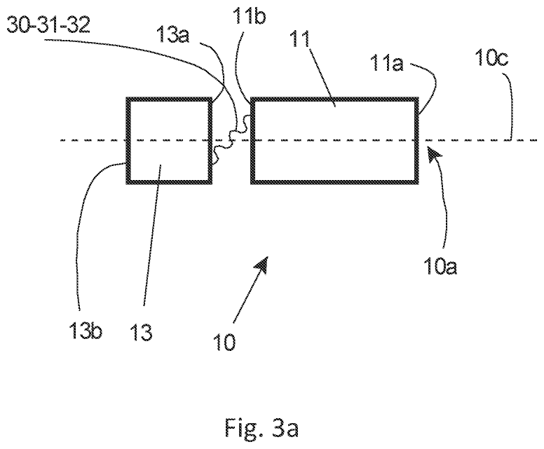

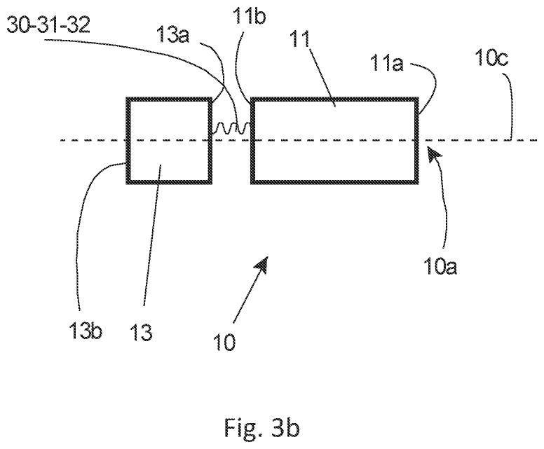

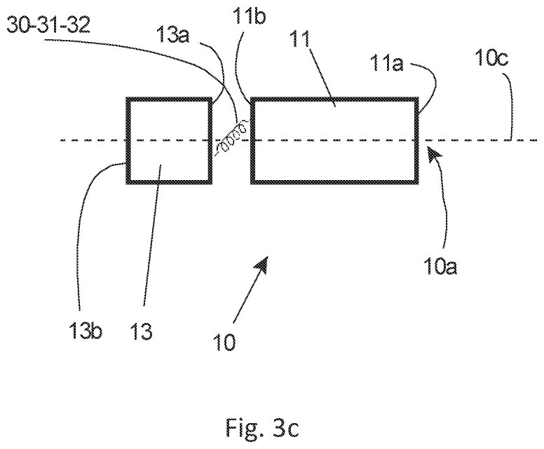

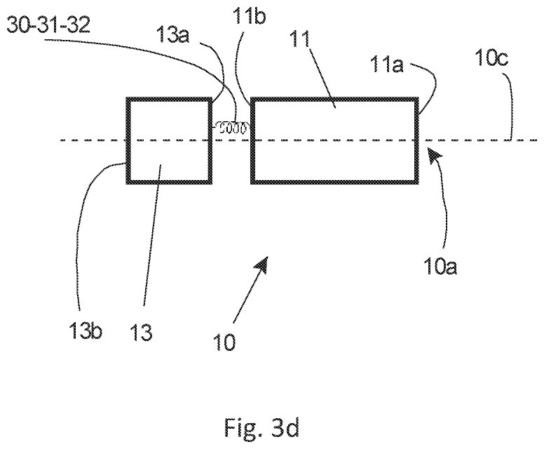

In the embodiments of the stent assembly 10 in both FIGS. 1a-1d and 2a-2d, the elongated filament rod elements 31-32, (31′-32′), (31″-32″) (31′″-32′″) have a zigzag structure, whereas in the embodiments of the stent assembly 10 in FIGS. 3a-3d, the elongated filament rod elements 31-32, (31′-32′), (31″-32″) (31′″-32′″) have a flexible winding or a coil structure. The zigzag, the winding as well as the coil embodiment allow a versatile extension of the elongated filament rod elements 31-32, (31′-32′), (31″-32″) (31′″-32′″) in their longitudinal direction thus setting within the vessel at its intended deployment location the translational distance between the adjacent stent segments being interconnected by these, either zigzag or coil formed, elongated filament rod elements 31-32 (31′-32′) (31″-32″) (31′″-32′″).

In the embodiments of the stent assembly 10 in FIGS. 1a-1d (zigzag version) as well as FIGS. 3a (windings version) and 3c (coil version), both the first rod end (31a-32a) and the second rod end (31b-32b) of the elongated filament rod elements 31-32 (31′-32′) (31″-32″) (31′″-32′″) are radially offset with respect to each other in both the first, initial length, configuration and the second, expanded length, configuration, seen in the direction of the longitudinal stent axis 10c.

In the embodiments of the stent assembly 10 in FIGS. 2a-2d (zigzag version) as well as in FIG. 3b (flexible winding version) and 3d (coil version), both the first rod end (31a-32a) and the second rod end (31b-32b) of the in length extendable elongated filament rod elements 31-32 (31′-32′) (31″-32″) (31′″-32′″) are longitudinally aligned with respect to each other in both the first, initial length, configuration and the second, expanded length, configuration, seen in the direction of the longitudinal stent axis 10c.

In FIGS. 1b and 2b, the initial minimum distance D1 between the proximal stent segment 11 and the first intermediate stent segment 13 as depicted in FIGS. 1a and 2a, has been adjusted to its maximum distance, now depicted with the reference numeral D3. The adjustment of the distance D1 between the proximal stent segment 11 and the first intermediate stent segment 13 from its initial, minimum distance D1 to its maximum distance D3, is established by simultaneous translation of the three intermediate stent segments 13-13′-13″, together with the distal stent segment 12 in the direction of (or along) the longitudinal stent axis 10c over its maximum distance relative to the proximal first stent segment 11 (which remains static and does not displace).

This simultaneous translation of the three intermediate stent segments 13-13′-13″, together with the distal stent segment 12 in the direction of (or along) the longitudinal stent axis 10c is depicted with the dashed two-ended arrow which encompasses the three intermediate stent segments 13-13′-13″, as well as the distal stent segment 12. The intermediate overall stent length Z′ now corresponds to the summation of the initial stent lengths X1, X2, X3 (triple), as well as three times the minimal distance D1 and a maximum distance D3. The elongated filament rod elements 31′-32′ interconnecting the proximal stent segment 11 with the first intermediate stent segment 13 are elongated till their full elongated length within the vessel, thus facilitating the translational elongation between the first stent segment 11 and the rest of the stent assembly 10 with the maximum distance D3.

Here it is to be noted that in an example of the deployment technique the first, proximal stent 11 is already inserted and deployed within the vessel, with the remainder of the stents 13-13′-13″-12 still accommodated in a compressed state within the catheter stent insertion device and ready for a next length adjustment as described below in relation to FIGS. 1c and 2c. Accordingly, the in length adjustable stent assembly according to the invention is capable of covering the complete ilio-femoral tract in a human or animal being, with the application/insertion of only one stent assembly. This has a significant benefit compared to already known stent placement procedures, wherein two separate stents are needed resulting in an unfavorable overlap. Accordingly, as the length of the stent assembly 10 according to the invention can be adjusted with respect of the intended location of deployment within the vessel, the adjustment and positioning of this in-situ in length adjustable stent assembly can be performed more precise using the phlebography and IVUS information collected during the procedure.

FIGS. 1c and 2c depicts the stent assembly 10, which, from its starting position shown in FIGS. 1b and 2b, has been extended further by adjusting the initial minimum distance D1 between the intermediate stent segment 13 and the intermediate stent segment 13′ (see FIG. 1c-2c) to an intermediate distance, now depicted with reference numeral D2 in FIGS. 1c and 2c. The adjustment of the distance between the first intermediate stent segment 13 and the second intermediate stent segment 13′ is accomplished by the simultaneous translation along the longitudinal stent axis 10c of the set of the second and third intermediate stent segments 13′ and 13″, together with the distal stent segment 12 relative to the proximal stent segment 11 and the first intermediate stent segment 13, which remain static and do not displace. The elongated filament rod elements 31″-32″ interconnecting the first intermediate segment 13 with the next intermediate stent segment 13′ are elongated till an intermediate elongated length, thus facilitating the translational elongation between the first intermediate stent segment 13 and the rest of the stent assembly 10 with an intermediate distance D2.

The resulting overall stent length Z″ is composed of the individual stent lengths X1, X2, and three times X3, as well as two minimal distances D1, one maximum distance D3 (as being set in the stent configuration shown in FIGS. 1b and 2b) and the intermediate distance D2.

Likewise, the next, proximal stent 13 is also already inserted and deployed within the vessel, with the remainder of the stents 13′-13″-12 still accommodated in a compressed state within the catheter stent insertion device and ready for a next length adjustment as described below in relation to FIGS. 1d and 2d.

FIGS. 1d and 2d shows yet another configuration of the stent assembly 10, which now exhibits an overall stent length of Z″. The new stent length Z″ has been created by a further extension step, wherein the third intermediate stent segment 13″, together with the distal stent segment 12, is being translated in the direction of (along) the stent axis 10c over its maximum translational distance relative to the proximal stent segment 11, the first and second intermediate stent segments 13-13′, which remain static and do not displace. The elongated filament rod elements 31′″-32′″ interconnecting the second intermediate segment 13′ with the next, here third and final, intermediate stent segment 13″ are elongated till the full, maximum elongated length, thus facilitating the translational elongation between the second intermediate stent segment 13′ and the rest of the stent assembly 10 with the maximum distance D3.

As such, the resulting overall stent length Z′″ is composed of the individual stent length X1, X2 and three times X3, as well as one minimum distance D1 (between the distal stent segment 12 and the third intermediate stent segment 13″), an intermediate distance D2 as being set in FIGS. 1c and 2c, as well as two times the maximum distance D3 as being set in FIGS. 1b-2b and 1 d-2d.

Here it is to be noted that the second next proximal stent 13′ is already inserted and deployed within the vessel, with the remainder of the stents 13″-12 still accommodated in a compressed state within the catheter stent insertion device.

It will be clear that with the subsequent translational displacement of parts of the stent assembly 10, that is the translation of all or less stent segments in the longitudinal direction of (along) the longitudinal stent axis 10c at any desired translational distance between the minimal distance D1 the maximum distance adjustment D3 relative to the static, unmovable already deployed stent segments, the distance between each adjacent stent segment can be adjusted at any desired intermediate distance between the minimum length D1 and the maximum length D3, the latter maximum length D3 corresponding with the maximum elongation of the flexible and elongated filament rods 31-32.

The amount of adjustment of the individual distances between the several adjacent stent segments can be arbitrarily chosen by the physician upon placement of the stent assembly 10 within the vessel, for example depending on the local constrictions within the vessel. As such, the individual locations of the several stent segments, in particular the intermediate stent segments 13-13′-13″ within the overall stent length, can be based on MR or CT imaging techniques, as well as real time fluoroscopy or intravascular ultrasound imaging, which are commonly used during stent placements.

FIGS. 4a, 4b and 4c show in more detail the length adjustment principle according to the invention. FIGS. 4a, 4b and 4c show enlarged views of the stent assembly 10 of FIGS. 1-3 in particular of the interconnection between two adjacent stent segments. Two adjacent stent segments are denoted with reference numerals 11 and 12 depicting a proximal stent segment 11 and a distal stent segment 12.

Both adjacent stent segments 11 and 12 are interconnected with each other using segment interconnection means or segment interconnection elements, denoted with reference numeral 30. In an example, the segment interconnection means or elements 30 comprise a first elongated filament rod 31 and a second elongated filament rod 32. However, for a proper operation of the invention also one elongated filament rod 31 suffices. In another embodiment, even three elongated filament rods can be implemented as being part of the segment interconnecting means 30

The elongated filament rod elements 31-32, here two rod elements, which are in length extendable, as outlined above. Both rod ends (31a-31b) (31b-32b) of the elongated filament rod elements 31-32 are longitudinally aligned with respect to each other in both the first, initial length, configuration and the second, expanded length, configuration, seen in the direction of the longitudinal stent axis 10c.

However also the interconnection principle shown in FIGS. 1a-1d, FIGS. 3a and 3c is possible, with both the first rod end (31a-32a) and the second rod end (31b-32b) of the elongated filament rod elements 31-32 being radially offset with respect to each other in both the first, initial length, configuration and the second, expanded length, configuration, seen in the direction of the longitudinal stent axis 10c

Additional intermediate stent segments 13-13′-13″-13′″-etc. can be positioned between the proximal stent segment 11 and the distal stent segment 12, similar as the stent assembly configurations of FIGS. 1a-1d and 2a-2d.

As explained above, both the first and second elongated filament rod 31 (32) are manufactured from an extendable material, for example from a flexible material, or the elongated filament rod element have in another example a telescopic structure also allowing extension in its longitudinal direction. In this example of FIGS. 4a-4c the elongated filament rod elements 31-32 have a zigzag structure. However, also the previously discussed flexible winding or coil structure are possible embodiments. They can be made from the same mesh material as the material of which the intermediate stent segments 13-13′-13″ as well as the proximal and distal stent segments 11 and 12 are made of. In an example, the material of the intermediate stent segments 13-13′-13″ and the proximal and distal stent segments 11 and 12 as well as of the segment interconnecting means 30 (the elongated filament rod) is Nitinol. Nitinol is a body-friendly metal memory material that, due to the external compression and mechanical forces to which stents, once implanted in a vessel, are subjected to, is used as the standard flexible stent material.

Each in length extendable elongated filament rod 31 (32) of the segment interconnecting means or elements 30 comprise a distal rod end 31b (32b) which is fixedly connected at connection point 12a1 (12a2) at the circumferential edge of the proximal segment end 12a of the stent segment 12. Likewise, each in length extendable elongated filament rod 31 (32) comprises at the opposite end of the rod a proximal rod end 31a (32a), which is likewise connected in a fixed manner at fixed locations, indicated as connection points 11b1 (11b2) at the circumferential edge of the distal segment face 11b of the proximal stent segment 11.

In this example the connection points 11b2 and 12a2 (and 11b1 and 12a1) are longitudinally aligned with respect to each other, seen in the direction of longitudinal stent axis 10c.

As it will be seen in FIG. 4a, both in length extendable elongated filament rods 31 and 32 exhibit their minimal length dimension, here due to their not yet expanded zigzag structure, similar as in FIGS. 1a and 2a. In the initial configuration wherein the distance D1 between both adjacent stent segments 11 and 12 is minimal, theoretically 0 (zero) mm, but in practice between 0.1-1.0 mm. The total initial length of the stent assembly 10 in FIG. 4a is denoted with L1, and corresponds with the two lengths of the stent segments 11 and 12 plus their initial, minimal intermediate distance D1.

According to the invention, the stent assembly 10 is allowed to extend along its longitudinal axis 10c during its deployment within the vessel by translating one of the stent segments (here stent segment 12) in the direction of (or along) the longitudinal stent axis 10c relative to the other of the two stent segments, here the proximal stent segment 11, which is kept in position by means of a (not shown) part of a catheter stent insertion device.

This translational principle within the vessel is shown in FIGS. 4b and 4c by means of the open arrow depicted next to the stent segment 12. By translation displacement of the stent segment 12 relative to the other stent segment 11, both in length extendable elongated filament rods 31 and 32 increase in their length dimension.

FIG. 4b depicts an intermediate configuration of the stent assembly 10, seen from the starting configuration depicted in FIG. 4a similar to e.g. FIG. 2b. It depicts an intermediate configuration of the two adjacent stent segments 11 and 12, the distance between both stent segments 11 and 12, denoted with D2, is larger than the initial minimum distance as denoted with reference numeral D1 in FIG. 4a. The total length of the stent assembly 10 in FIG. 4b is denoted with L2 and corresponds with the two lengths of the stent segments 11 and 12 plus the now increased intermediate distance D2, due to the length increase of both in length extendable elongated filament rods 31 and 32.

Further translation of the distal stent segment 12 along the longitudinal stent axis 10c relative to the proximal stent segment 11 results in a further increase of the distance between both intermediate stent segments 13 and 13′ due to a further length increase of the in length extendable elongated filament rods 31 and 32. This translational extension ends until the maximum length extension of the elongated filament rods 31 and 32 and thus the maximum distance between both intermediate stent segments is reached. This configuration is depicted in FIG. 4c, wherein the elongated filament rods 31 and 32 are fully extended in length within the vessel and the distance between both stent segments 11 and 12 has reached its maximum length and is denoted with the reference numeral D3. The total length of the stent assembly 10 in FIG. 4c is now L3 and corresponds with the two lengths of the stent segments 11 and 12 plus the maximum intermediate distance D3, due to the maximum length increase of both in length extendable elongated filament rods 31 and 32.

It is clear that the maximum distance D3 between both stent segments 11 and 12 depends on the maximum expansion length of the in length extendable flexible elongated filament rods 31 and 32, which interconnect both stent segments 11 and 12. Their maximum expansion length is in part dependent on the flexible material chose for the flexible elongated filament rods 31 and 32 as well as its zigzag, multiple winding or coil structure. In FIG. 4c, depicting the maximum extension, both elongated filament rods 31 and 32 exhibit a maximum elongation in a parallel orientation relative to the longitudinal stent axis 10c.

Thus, it is to be noted that the stent elongation principle within a vessel as depicted in FIGS. 1-4 is meant as an example and is not to be considered the only possibility to lengthen a stent assembly according to the invention, that is consisting of five stent segments. Any distance between either adjacent stent segments can be altered and elongated to any distance between its minimal (D1) and maximal value (D3) by means of translational elongation using in length extendable elongated filament rods, which can be extended in length, either by a suitable flexible material and/or geometrical structure such but not limited to the zigzag, the multiple windings, the coil or telescopic structure.

The distance D1 theoretically equals 0 (zero) mm, but in practice the minimal value of D1 is approx. 0.5-1.0 mm. Similarly one or more distances between adjacent stent segments can remain unaltered (stay at their minimal length D1), in fact they can be skipped, whereas a specific distance is to be changed, upon decision by the physician, who decides on the ultimate stent lengthening based on the local restrictions within the vessel near the intended deployment position of the stent assembly 10.

With the stent elongation mechanism as described in this patent application, the physician can easily adapt the stent length during its stent deployment within the vessel and in particular set the location of a specific intermediate stent segment within the overall stent assembly, such that each intermediate stent segment abuts and supports several desired locations of the vessel wall after insertion and deployment.

As stated above in an example of the deployment technique each next proximal stent is already inserted and deployed within the vessel, with the remainder of the stents still accommodated in a compressed state within the catheter stent insertion device and arranged for a next length adjustment. Once the physician is of the opinion that the overall stent assembly 10 has the correct length and the correct initial proximal position within the vessel, the decision is made to insert and deploy the remainder of the stent segments within the vessel under simultaneous withdrawal in the distal direction of the catheter stent insertion device. As such the complete stent assembly 10 with the correct, adjusted length will be deployed within the vessel covering the correct vessel length as intended.

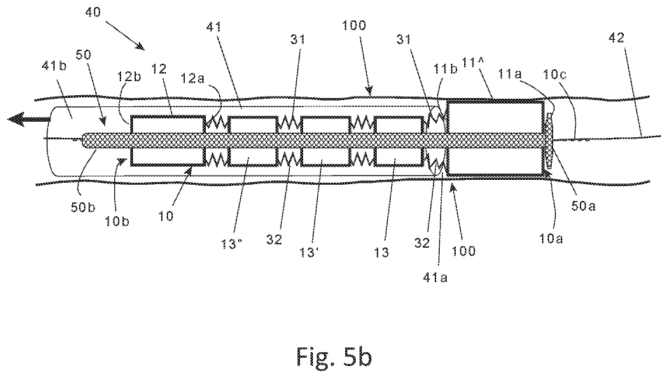

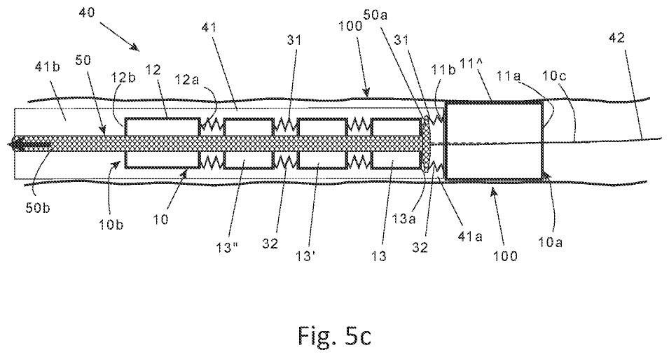

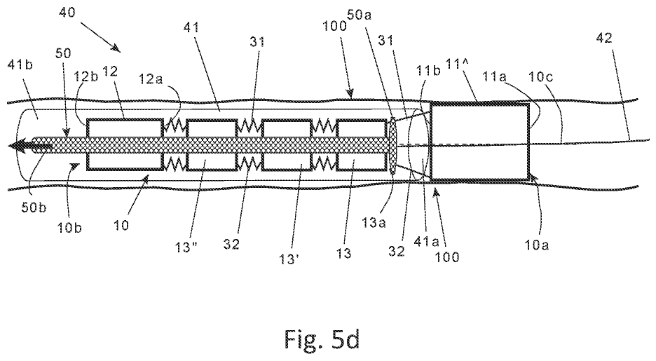

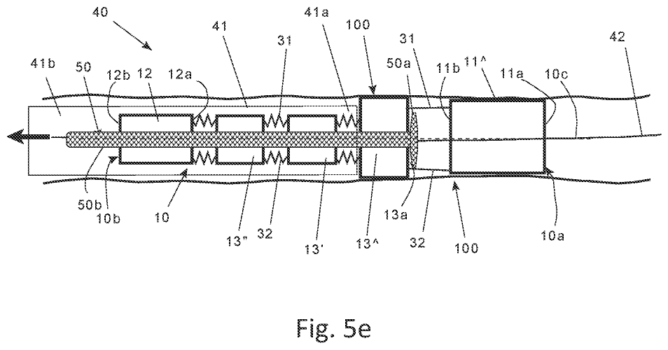

In FIGS. 5a-5e a catheter stent insertion device 40 according to the invention is disclosed in a sequence of operational stages of the deployment of a stent assembly 10 according to the invention in a vessel 100. The catheter stent insertion device 40 is schematically depicted and is composed of a hollow stent accommodating tube 41 for accommodating the stent (also indicated as the stent assembly) 10 in a compressed configuration. The hollow stent accommodating tube 41 has a proximal tube end 41a (depicted at the right side of the page of FIG. 5) which is open, as well as a distal tube end 41b (depicted at the left side of the page of FIGS. 5a-5e). The distal tube end 41b is connected to guidance means (not depicted) which are positioned outside the human body.

Reference numeral 42 denotes a guide wire which is deployed in the vessel 100 for guiding the catheter stent insertion device 40 during the several stent deployments stages. In this example of stent deployment, the stent assembly 10 is composed of a proximal stent segment 11, three intermediate stent segment 13-13′-13″ and a distal stent segment 12. The several stent segments 11-13-13′-13″-12 are interconnected with each with interconnecting means 30 composed of the several in length extendable elongated rod elements 31-32 according to the embodiment shown in FIGS. 2a-2d and FIGS. 4a-4c, being the zigzag variant which longitudinally aligned with respect to each other in both the first, initial length, configuration and the second, expanded length, configuration, seen in the direction of the longitudinal stent axis 10c.

It is noted that the flexible winding or a coil structure variant is equally suitable for setting the translational distance between the adjacent stent segments being interconnected by these, either zigzag, flexile winding or coil formed, elongated filament rod elements 31-32. Similarly, implementation of the elongated filament rod elements 31-32 being radially offset with respect to each other in both the first, initial length, configuration and the second, expanded length, configuration, seen in the direction of the longitudinal stent axis 10c as shown in FIGS. 1a-1d is also possible as a stent assembly for use in a catheter stent insertion device 40 according to the invention.

The catheter stent insertion device 40 is to be inserted with its proximal tube end 41a inside the vessel 100 towards the intended deployment location. At said location the stent (assembly) 10 is to be deployed, such that after deployment within the vessel the separate stent segments 11-13-13′-13″-12 expand and abut against the inner vessel wall.

The catheter stent insertion device 40 also comprises translational lengthening means depicted with reference numeral 50, which are, in this example, constructed as a spindle 50. The translational spindle 50 is in essence made from a rigid bar-like element, for example from a rigid plastic material. The translational spindle 50 is accommodated inside the hollow stent accommodating tube 41 and is also accommodated inside the hollow cylindrically configured yet compressed stent assembly 10.

It is to be noted that FIGS. 5a-5e depicts the catheter stent insertion device 40 in an enlarged view and it should be clarified that the dimensions of the several parts are such that the stent assembly 10 in its compressed configuration is closely placed around the translational spindle 50 and is also closely encapsulated within the hollow stent accommodating tube 41. As such the miniaturized configuration allows the insertion inside a vessel of a human or an animal body.

The translational spindle 50 is provided at its proximal end with an enlarged stent engagement end 50a. As the translational spindle 50 is accommodated inside the hollow stent accommodating tube 41, the enlarged stent engagement end 50a has an outer dimension which is smaller than the inner dimension of the hollow stent accommodating tube 41, thus allowing translational displacement of the whole translation spindle 50 within the hollow stent accommodating tube 41 along the longitudinal orientation of the hollow stent accommodating tube 41, along the guide wire 42 and the stent assembly 10 and the vessel 100 (said longitudinal orientation denoted with reference numeral 10c). However, for effectuating the translational lengthening of the stent assembly 10—as outlined in the FIGS. 1a-1d, 2a-2d and 4a-4c, the outer dimension of the enlarged stent engagement end 50a is larger, preferably slight larger than the outer dimension of each individual stent segment 11-13-13′-13″-12 of the stent assembly 10, albeit in their compressed, not yet deployed configuration. Furthermore, the outer dimension of the enlarged stent engagement end 50a is smaller than the inner dimension of each individual stent segment 11-13-13′-13″-12 of the stent assembly 10, in their expanded, deployed configuration.

FIG. 5a depicts the initial stage of the catheter stent insertion device 40 with the compressed stent assembly 10 accommodated around the translation spindle 50 and both completely accommodated in the hollow stent accommodating tube 41. The enlarged stent engagement end 50a is aligned with or closely positioned near the proximal tube end 41a. The enlarged stent engagement end 50a abuts the proximal end 10a of the compressed stent assembly 10 in particular abuts against the proximal segment face 11a of the compressed first, proximal stent segment 11. The catheter stent insertion device 40 is advanced through the vessel 100 using the guide wire 42 until proximal device end 41a of the hollow stent accommodating tube 41 has reached the position of deployment of the proximal end 10a of the stent assembly 10.

The manipulation and advancement of the catheter stent insertion device towards its desired or intended deployment position within the vessel 100 can be performed by means of guidance means positioned outside the patient, or by hand by the physician, as denoted with the black arrow pointing towards the right (=proximal direction) in FIG. 5a, next to the proximal device end 41a. In either method (automated or by hand) the location of the (proximal device end 41a of the) catheter stent insertion device 40 prior to deployment of the stent 10 can be checked under fluoroscopy or with any other known imaging technique being used with stent placement.

Deployment of the first, proximal stent segment 11 is shown in FIG. 5b, takes place by the translation retraction of the hollow stent accommodating tube 41 along the longitudinal direction 10c in distal direction, as depicted with the black arrow pointing towards the left next to the distal device tube end 41b. During the translation retraction of the hollow stent accommodating tube 41, the stent assembly 10 as well as the translation spindle 50 remain in their initial deployment position. When the hollow stent accommodating tube 41 is retracted over a translational distance at least equal to the length of the proximal stent segment 11 (denoted with X1 in FIGS. 1a-1d, and 2a-2d), the first, proximal stent segment 11 is released via the open proximal tube end 41a within the vessel 100 at the desired or intended deployment location, after which the stent segment 11 deploys and expands and abuts against the inner vessel wall of the vessel 100. This deployment configuration of stent segment 11 is shown in FIG. 5b with reference numeral 11\hat{ }.

Referring to FIG. 5c, the outer dimension of the enlarged stent engagement end 50a is smaller than the inner dimension of each individual stent segment 11-13-13′-13″-12 of the stent assembly 10, in their expanded, deployed configuration. In a further deployment step, the translation spindle 50 is retracted in the distal direction within the hollow stent accommodating tube 41 and through the expanded stent segment 11\hat{ } until the enlarged stent engagement end 50a is aligned again with or closely positioned near the proximal tube end 41a, which has been retracted in the distal direction earlier (FIG. 5b). The enlarged stent engagement end 50a now abuts against the proximal segment face 13a of the next, yet compressed, stent segment 13.

In FIG. 5c, the first and second elongated filament rods 31-32 exhibit still their initial length dimension.

In FIG. 5d, showing a stent lengthening step, the translation spindle 50 is further retracted in the distal direction (see the black arrow next to the distal spindle end 50b and pointing to the left=distal direction). As the outer dimension of the enlarged stent engagement end 50a is smaller than the outer dimension of each individual stent segment 11-13-13′-13″-12 of the stent assembly 10 in their compressed configuration, the translation spindle 50 will likewise pull or displace the rest of the stent assembly, here the stent segments 12-13″-13′-13, in the distal direction and away from the first stent segment 11, which is already deployed and anchored against the inner vessel wall of the vessel 100.

The larger enlarged stent engagement end 50a abuts against the smaller proximal segment face 13a of the compressed stent segment 13 and pulls the rest of the compressed stent assembly, here the compressed stent segments 12-13″-13′-13, inside and relative to the hollow stent accommodating tube 41—which remains at its location within the vessel—in the distal direction, causing a translation lengthening of the first and second elongated filament rods 31-32 as explained above in connection with FIGS. 1a-1d, 2a-2d and 4a-4c.

In FIG. 5d, the translation spindle 50 and the compressed stent segments 12-13-13′-13″ are displaced in the distal, translational direction over a distance causing the maximum lengthening the first and second elongated filament rods 31-32 from their minimal distance (D1 in the FIGS. 1a-1d, 2a-2d and 4a-4c) to the maximal distance D3. However, for effectuating any desired translational lengthening of the first and second elongated filament rods 31-32 interconnecting an already deployed and expanded stent segment with a not-yet deployed and still compressed stent segment within the hollow stent accommodating tube 41, the distal displacement of the translation spindle 50 and subsequent ‘pulling’ at the assembly of compressed stent segments still present in the hollow stent accommodating tube 41 can be interrupted with the use of suitable imaging techniques. Herewith the lengthening of the elongated filament rods 31-32 can be set at any length between the minimum distance D1 and the maximum distance D3.

With the intermediate distance between the already deployed and expanded proximal stent segment 11 and the not-yet deployed and still compressed stent segment 13 being set at the maximum distance D3 in FIG. 5d, deployment of this still compressed stent segment 13 is shown in FIG. 5e.

Deployment takes place in a similar manner as in FIG. 5b, by the translation retraction of the hollow stent accommodating tube 41 along the longitudinal direction 10c in distal direction, as depicted with the black arrow pointing towards the left next to the distal device tube end 41b. During the translation retraction of the hollow stent accommodating tube 41, the remaining compressed stent segments 12-13″-13′-13, the translation spindle 50 as well as the already deployed stent segment 11 remain in their position

When the hollow stent accommodating tube 41 is retracted over a translational distance at least equal to the length of the stent segment 13 (denoted with X3 in FIGS. 1a-1d, and 2a-2d), the intermediate stent segment 13 is released via the open proximal tube end 41a within the vessel 100 at the desired or intended deployment location next to and at a distance D3 from the already deployed stent segment 11. Similarly, after deployment, the intermediate stent segment 13 deploys and expands and abuts against the inner vessel wall of the vessel 100 (denoted with 13\hat{ } in FIG. 5e).

The steps for setting the distance between the now deployed intermediate stent segment 13 and the next intermediate stent segment 13′ are repeated and are similar as explained with reference to FIGS. 5c and 5d.

With the catheter stent insertion device 40 of FIGS. 5a-5e each individual next stent segment can be positioned at a desired distance from an already deployed stent segment. By setting the intermediate distances and deploying the stent segments one by one within the vessel by a translational displacement of the translation spindle 50 in the distal direction towards the distal device end 41a a stent assembly 10 according to the invention can be effectively deployed within a vessel. After deployment of one of the stent segments, the remainder of the stents still accommodated in a compressed state within the hollow stent accommodating tube 41 of the catheter stent insertion device 40 can be subjected to a next length adjustment relative to the next deployed stent segment by the subsequent translation of the translation spindle 50 in the distal direction over any desired length of the relevant interconnecting elongated filament rods 31-32.

### REFERENCE NUMERAL LISTING

10 stent
10a proximal stent end of stent 10
10b distal stent end of stent 10
10c longitudinal axis of stent 10
11 first, proximal stent segment
11\hat{ } first, proximal stent segment in expanded configuration
11a proximal end of proximal stent segment
11b distal end of proximal stent segment
11b1 first connection between distal end 11b of stent segment 11 and proximal rod end
31a of first elongated filament rod 31
11b2 second connection between distal end 11b of stent segment 11 and proximal rod end 32a of second elongated filament rod 32
12 second, distal stent segment
12a proximal end of distal stent segment
12a1 first connection between proximal end 12a of stent segment 12 and distal rod end
31b of first elongated filament rod 31
12a2 second connection between proximal end 12a of stent segment 12 and distal rod end 32b of second elongated filament rod 32
12b distal end of distal stent segment
13 first intermediate stent segment
13\hat{ } first intermediate stent segment in expanded configuration
13a proximal end of intermediate stent segment
13b distal end of intermediate stent segment
13′ second intermediate stent segment
13″ third intermediate stent segment
30 segment interconnecting means
31 first elongated filament rod
31a proximal rod end of first elongated filament rod
31b distal rod end of first elongated filament rod
32 second elongated filament rod
32a proximal rod end of second elongated filament rod
32b distal rod end of second elongated filament rod
D1 initial, minimal distance between two intermediate stent segments
D2 intermediate distance between two intermediate stent segments
D3 maximum distance between two intermediate stent segments
X1 length of first, proximal stent segment 11
X2 length of second, distal stent segment 12
X3 length of intermediate stent segment 13-13′-13″
Z initial length of the stent of FIG. 1
Z′ an intermediate length of the stent of FIG. 2
Z″ a further intermediate length of the stent of FIG. 3
Z′″ a further intermediate length of the stent of FIG. 4
40 catheter stent insertion device
41 hollow stent accommodating tube
41a open proximal tube end
41b distal tube end
42 guide wire
50 lengthening means or translation spindle
50a enlarged stent engagement end of translation spindle 50
50b distal spindle end
100 vessel

1. A stent for insertion in a vessel of a human or animal body, said stent having a proximal end, a distal end and a longitudinal stent axis, and comprising at least two stent segments, as well as segment interconnecting means interconnecting two stent segments, wherein said segment interconnecting means are structured in adjusting a distance between said two stent segments when inserted in a vessel between a first configuration, wherein said distance is minimal and a second configuration, wherein said distance is maximal, due to a translation displacement along said longitudinal stent axis of one of said two stent segments relative to the other of the two stent segments.

2. The stent for insertion in a vessel of a human or animal body according to claim 1, wherein, for interconnecting said at least two stent segments, said segment interconnecting means comprise at least one elongated filament rod having two rod ends, a first rod end being connected to the first stent segment and the second rod end being connected to the second stent segment, wherein a distance between both first and second rod ends is smaller in said first configuration than in said second configuration.

3. The stent for insertion in a vessel of a human or animal body according to claim 1, wherein in said first configuration and said second configuration said first rod end and said second rod end are radially offset with respect to each other.

4. The stent for insertion in a vessel of a human or animal body according to claim 1, wherein in said first configuration and said second configuration said first rod end and said second rod end are longitudinally aligned with respect to each other, seen in the direction of said longitudinal stent axis.

5. The stent for insertion in a vessel of a human or animal body according to claim 1, wherein said at least one elongated filament rod is structured to extend in length.

6. The stent for insertion in a vessel of a human or animal body according to claim 5, wherein said at least one elongated filament rod is structured to irreversible or reversible extend in length.

7. The stent for insertion in a vessel of a human or animal body according to claim 5, wherein said at least one elongated filament rod is manufactured from an extendable material, the extendable material including a flexible material.

8. The stent for insertion in a vessel of a human or animal body according to claim 5, wherein said at least one elongated filament rod has a telescopic structure.

9. The stent for insertion in a vessel of a human or animal body according to claim 5, wherein said at least one elongated filament rod has a zigzag structure.

10. The stent for insertion in a vessel of a human or animal body according to claim 5, wherein said at least one elongated filament rod has a coil structure.

11. The stent for insertion in a vessel of a human or animal body according to claim 1, wherein said stent comprises a proximal stent segment, a distal stent segment and one or more intermediate stent segments disposed between the proximal and distal stent segments, and wherein said segment interconnecting means interconnect each of said stent segments.

12. The stent for insertion in a vessel of a human or animal body according to claim 11, wherein, seen along said longitudinal axis of the stent, said proximal stent segment has a first length, said distal stent segment has a second length, and said intermediate stent segments have a third length, wherein said third length is smaller than said first and second length.

13. The stent for insertion in a vessel of a human or animal body according to claim 12, wherein said third length is 5-15 mm.

14. The stent for insertion in a vessel of a human or animal body according to claim 12, wherein said first length and said second length are the same.

15. The stent for insertion in a vessel of a human or animal body according to claim 12, wherein said first length is longer than said second length, wherein said first length is 30-50 mm and said second length is 10-30 mm.

16. The stent for insertion in a vessel of a human or animal body according to claim 1, wherein said maximal distance between said stent segments is between 1 mm.

17. The stent for insertion in a vessel of a human or animal body according to claim 1, wherein the number of said intermediate stent segments is between 1-30.

18. A catheter stent insertion device for inserting a stent assembly composed of at least two stent segments according to claim 1, in a vessel of a human or animal body, said catheter stent insertion device at least comprising:
a hollow stent accommodating tube having an open proximal tube end and a distal tube end, said hollow stent accommodating tube being arranged for accommodating said stent assembly in a compressed configuration, as well as
lengthening means arranged in adjusting within the hollow stent accommodating tube a distance between an already deployed stent segment and a next interconnected stent segment between a first configuration, wherein said distance is minimal and a second configuration, wherein said distance is maximal, by translating the next interconnected stent segment along the longitudinal stent axis relative to the already deployed stent segment.

19. The catheter stent insertion device according to claim 18, wherein said lengthening means comprises a translation spindle extending through the stent assembly in its compressed configuration, said translation spindle comprising an enlarged distal stent engagement end having an outer dimension larger than an outer dimension of a stent segment in its compressed configuration.

20. The catheter stent insertion device according to claim 19, wherein the enlarged distal stent engagement end has an outer dimension smaller than an inner dimension of a stent segment in its expanded, deployed configuration.

21. The catheter stent insertion device according to claim 18, further comprising guidance means for guiding said hollow stent accommodating tube with its proximal tube end towards a deployment location within said vessel.

22. A method for inserting a stent assembly according to the invention at a deployment location within a vessel of a human or animal body using a catheter stent insertion device according to claim 1, the method comprising the steps of:
A inserting the catheter stent insertion device accommodating said stent assembly composed of at least two stent segments in a compressed configuration in said hollow stent accommodating tube with its open proximal tube end in the vessel;
B guiding the catheter stent insertion device towards said deployment location within the vessel;
C retracting said hollow stent accommodating tube until the proximal one of the at least two stent segments is deployed via the open proximal tube end in the vessel;
D retracting one of said at least two stent segments along the longitudinal stent axis relative to the other already deployed one of the at least two stent segments, thereby adjusting a distance between said at least two stent segments between a first configuration, wherein said distance is minimal and a second configuration, wherein said distance is maximal;
E retracting said hollow stent accommodating tube and deploying the next one of the at least two stent segments via the open proximal tube end in the vessel.

23. The method of claim 22, further characterized by the step of:
D1 repeating step D for each next individual stent segment of the stent.

24. The method of claim 23, further characterized by the step of:
performing steps D and D1 for each of the individual stent segments within the hollow stent accommodating tube prior to step C.
